(19) United States
(12) Patent Application Publication
(10) Pub. No.: US 2006/0115468 A1
(43) Pub. Date: Jun. 1, 2006

(54) Dietary supplement for treating and preventing gastrointestinal disorders

(76) Inventor: Kara Morrison, Chillicothe, OH (US)

Correspondence Address: THE INVENTORS NETWORK, INC., 332 ACADEMY STREET, CARNEGIE, PA, 15106, US

(21) Appl. No.: 10/997,655

(22) Filed: Nov. 26, 2004

(51) Int. Cl. A61K 38/47; A61K 38/46; A61K 36/82; A61K 36/736; A61K 36/752; A61K 36/68; A61K 36/534; A61K 36/899; A61K 36/906; A61K 36/28; A61K 36/48; A61K 36/73

(52) U.S. Cl. 424/94.61; 424/94.65; 424/725; 424/757; 424/735; 424/736; 424/764; 424/750; 424/765; 424/747; 424/738; 424/195.17; 424/768; 424/769; 424/729; 424/766; 424/756

(57) ABSTRACT

A dietary and vitamin supplement, in capsule or powder form, for enhancing and maintaining digestive health and colon care, and alleviating gastrointestinal disorders, includes a blend of natural ingredients, including acidophilus, digestive enzymes and antioxidants, and which can be taken on an as-needed basis, in either capsule or liquid form, along with any other medications prescribed for the individual.

### FIELD OF THE INVENTION

The present invention pertains to dietary and vitamin supplements, and more particularly pertains to a dietary and vitamin supplement for promoting gastrointestinal care and health that combines natural ingredients, digestive enzymes and antioxidants in an orally administered form.

### BACKGROUND OF THE INVENTION

Gastrointestinal disorders are widespread due to numerous cultural, environmental and dietary factors. Among the cultural and environmental factors are the work stresses and the sedentary nature of daily life that affect the body's metabolism including proper gastrointestinal functioning. These stresses often lead to the excessive intake of tobacco, alcohol and illegal drugs—all of which have pernicious effects on gastrointestinal functioning. In addition, modem dietary habits that both accompany and are a consequence of the aforementioned factors, contribute to many gastrointestinal disorders. Thus, the consumption of fatty, greasy, calorie-laden fast food is a dominant dietary cause of many gastrointestinal disorders.

One common way to maintain gastrointestinal health is to partake of a high fiber diet. Fibrous diets have many salutary effects that include providing bulk to the stool, decreasing intestinal transit time, and decreasing the ph content of the gastrointestinal tract. Also, a fibrous diet decreases the number of pernicious oxygen free radicals circulating in the body.

In addition, certain bacteria have demonstrated the ability to improve and maintain gastrointestinal health. For example, bacteria of the genus Lactobacilus have been used for several hundred years in treating various gastrointestinal disorders. Modern biotechnology techniques have greatly increased the types and strains of Lactobacilus that can be used in the broad or narrow range spectrum of treatments for gastrointestinal disorders. Thus, the prior art reveals a diversity of treatments and compositions for gastrointestinal disorders.

For example, the Reddy patent (U.S. Pat. No. 4,806,368) discloses a nutritional supplement in which the primary ingredients are Lactobacillus acidophilus, vitamins A, C, D and E, mineral supplements, apple fiber, L-cystine and lecithin, and which is especially useful in treating yeast-related problems.

The Bounnous et al. patent (U.S. Pat. No. 5,290,571) discloses an undenatured whey protein composition obtained from raw bovine, goat or sheep milk, and which produces a number of enhanced immunological effects on various types of infections and carcinomas.

The Brassart et al. patent (U.S. Pat. No. 5,578,302) discloses a treatment for stomach ulcers that includes an orally administered composition primarily containing Lactobacillus johnsoni strain CNCM I-1225.

The Izvekova et al. patent (U.S. Pat. No. 6,156,320) discloses anti-bacterial products produced from cultures or ferments that can be cultured in milk, and that in a preferred embodiment comprises the Lactobacillus acidophilus strain N.V. Er 317/402.

The Patton et al. patent (U.S. Pat. No. 5,997,848) discloses an insulin delivery system in which the inhalation of a dry powder of insulin is delivered to the alveolar regions of the lungs.

The Paul patent (U.S. Pat. No. 6,180,099 B1) discloses both methods of use and compositions for improving gastrointestinal health, with the composition primarily consisting of an immunoglobulin and a soluble dietary fiber.

The Paul et al. patent (U.S. Pat. No. 6,241,983 B1) discloses a composition for human gastrointestinal health that includes a beneficial human intestinal microorganism, dietary fiber, and, optionally, an immunoglobulin composition.

Nonetheless, despite the ingenuity of the above compositions, there remains a need for a dietary and vitamin supplement that promotes healthy gastrointestinal care and includes natural ingredients, digestive enzymes and antioxidants.

### SUMMARY OF THE INVENTION

The present invention comprehends a dietary and vitamin supplement, to be administered orally by capsule or in a powdered form for dilution in a liquid such as a juice drink, that promotes and maintains positive colon care and digestive health for the avoidance and prevention of the various gastrointestinal disorders.

The present invention is a composition that includes various natural ingredients, digestive enzymes and antioxidants with the supplement being taken on an as-needed basis without a doctor's prescription. A primary component of the natural ingredients is Acidophilus, and the supplement can accompany the use of any normally prescribed medications.

It is an objective of the present invention to provide a dietary and vitamin supplement that enhances colon care and digestive health.

It is another objective of the present invention to provide a dietary and vitamin supplement that can be administered in an easy-to-swallow oral form.

It is yet another objective of the present invention to provide a dietary and vitamin supplement that can be reduced to a powdered form for admixing with a liquid, such as a juice drink, to enhance the palatability of the supplement.

It is still yet another objective of the present invention to provide a dietary and vitamin supplement that synergistically combines natural ingredients, digestive enzymes and antioxidants to promote and enhance a healthy colon and digestive system.

Still yet another objective of the present invention is to provide a dietary and vitamin supplement that allows the individual to easily treat and prevent numerous digestive and stomach ailments and debilities.

These and other objects, features and advantages will become apparent to those skilled in the art upon a perusal of the following detailed description read in conjunction with the accompanying drawing figures.

### BRIEF DESCRIPTION OF THE DRAWINGS

FIG. 1 is a perspective view of the dietary and vitamin supplement showing the containers for the supplement and the capsule form of the supplement; and

FIG. 2 is a plan view listing the ingredients for the dietary and vitamin supplement of the present invention.

### DETAILED DESCRIPTION OF THE PREFERRED EMBODIMENT

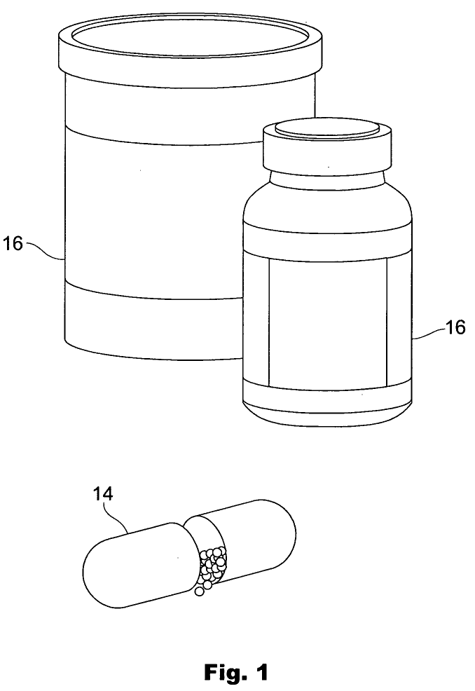

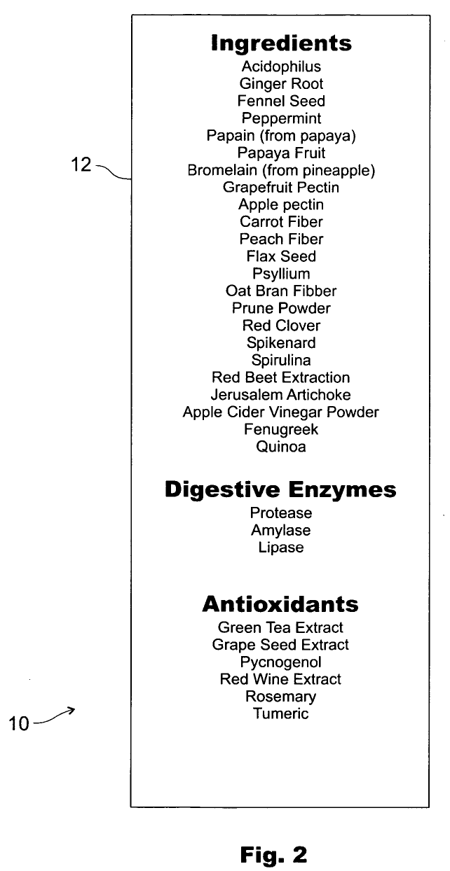

Illustrated in FIGS. 1 and 2 is a dietary and vitamin supplement 10 for supporting and maintaining good digestive health and colon care, and for the alleviation and prevention of numerous gastrointestinal ailments and disorders. A notable gastrointestinal disorder the supplement 10 of the present invention is designed to treat and prevent is irritable colon, or colitis that is the inflammation of the mucous membrane of the colon.

Thus, as shown in FIGS. 1 and 2, the dietary and vitamin supplement 10 is composed of a range of natural ingredients that function when ingested and dispersed within the body to promote and maintain positive digestive health and colon care. The natural ingredients list 12 as shown in FIG. 2 starts with Acidophilus, a type of bacteria that thrives in an acid medium or in dilute acid, and is thus commonly used in the treatment and relief of gastrointestinal ailments and disorders. In addition to the Acidophilus, the following other ingredients are included in the composition: ginger root, fennel seed, peppermint, papain (from papaya), papaya fruit, bromelain (from pineapple), grapefruit pectin, apple pectin, carrot fiber, peach fiber, flaxseed, pysllium, oat bran fiber, prune powder, red clover, spikenard, spirulina, red beef extraction, Jerusalem artichoke, apple cider vinegar powder, fenugreek and Quinoa. The composition also includes a number of digestive enzymes prominent among them being protease, amylase, lipase.

As illustrated in FIG. 2, the composition list 12 also includes antioxidants that combat and neutralize the effect of free radicals on the body's systems, including the gastrointestinal system. The antioxidants include green tea extract, grape seed extract, pycnogenol, red wine extract, Rosemary, and Tumeric.

The above-described composition can be developed into an easy-to-swallow gel capsule form 14, as shown in FIG. 1, for storage and dispensing from variously sized containers 16; or the supplement can be developed into a powder form for dilution and consumption in a liquid, such as a fruit or juice drink.

Although the foregoing invention has been described in detail by way of illustration and example, for purposes of clarity of understanding, it will be obvious to those skilled in the art the numerous modifications, alterations, and variations will be possible and practicable, while still remaining within the spirit of the invention and the scope of the appended claims.

I claim:

1. A dietary and vitamin supplement for treating and alleviating gastrointestinal disorders, comprising:
natural ingredients selected from the following group:
acidophilus;
ginger root;
fennel seed;
peppermint:
papain that is derived from papaya;
bromelain that is derived from pineapple;
grapefruit pectin;
apple pectin;
carrot fiber;
peach fiber;
flax seed;
psyllium;
oat bran fiber;
prune powder;
red clover;
spikenard;
spirulina;
red beet extraction;
Jerusalem artichoke;
apple cider vinegar powder;
fenugreek;
quinoa;
digestive enzymes selected from the following group:
protease;
amylase;
lipase;
antioxidants selected from the following group:
green tea extract;
grape seed extract;
pycnogenol;
red wine extract;
rosemary; and
tumeric.

2. The dietary and vitamin supplement of claim 1 wherein the supplement can be delivered in a capsule form.

3. The dietary and vitamin supplement of claim 2 wherein the supplement can be delivered in a powder form for dilution in a liquid.

4. A dietary and vitamin supplement for treating and alleviating gastrointestinal disorders, comprising:
natural ingredients, digestive enzymes, and antioxidants that include:
acidophilus;
ginger root;
fennel seed;
peppermint;
papain derived from papaya;
papaya fruit;
bromelain derived from pineapple;
grapefruit pectin;
apple pectin;
carrot fiber;
peach fiber;
flax seed;
psyllium;
oat bran fiber;
prune powder;
red clover;
spikenard;
spirulina;
red beet extraction;
Jerusalem artichoke;
apple cider vinegar powder;
fenugreek;
quinoa;
protease;
amylase;
lipase;
green tea extract;
pycnogenol;
red wine extract;
rosemary; and
tumeric.
